(19) United States
(12) Patent Application Publication
(10) Pub. No.: US 2016/0106776 A1
(43) Pub. Date: Apr. 21, 2016

(54) FEED SUPPLEMENT AND ITS USE

(71) Applicant: DSM IP ASSETS B. V., Heerlen (NL)

(72) Inventors: Catherine HAMELIN, Basel (CH); Rual LOPEZ ULIBARRI, (US); Wolfgang SCHLIFFKA, Basel (CH)

(73) Assignee: DSM IP ASSETS B.V., Heerlen (NL)

(21) Appl. No.: 14/893,237

(22) PCT Filed: May 2, 2014
(86) PCT No.: PCT/EP2014/058967

§ 371 Date: Nov. 23, 2015

(30) Foreign Application Priority Data
May 31, 2013 (EP) 13169984.5

(51) Int. Cl. A61K 33/04; A61K 31/355; A61K 31/085; A61K 31/05; A61K 31/015; A61K 31/11; A61K 31/122; A61K 31/375

(52) U.S. Cl. CPC A61K 33/04; A61K 31/122; A61K 31/355; A61K 31/375; A61K 31/05; A61K 31/015; A61K 31/11; A61K 31/085; A23K 1/1603; A23K 1/1606; A23K 1/164; A23K 1/1758; A23K 1/1826

(57) ABSTRACT

The present invention relates to feed supplements comprising vitamins and canthaxanthin and the use of such supplements for improving performance and health in poultry, in particular in poultry pullets. It has been found surprisingly that feed additive compositions as hereinafter defined improves immune status, bone and skeletal development, flock uniformity, growth and feed conversion of pullets. The feed additive composition according to the present invention comprises canthaxanthin, at least one vitamin selected from the group consisting of vitamin C and vitamin E, a trace mineral, in particular selenium, and optionally a mixture of at least two compounds selected from the group consisting of thymol, eugenol, vanillin and γ-terpinene.

The present invention relates to feed supplements comprising vitamins and canthaxanthin and the use of such supplements for improving performance and health in poultry, in particular in poultry pullets.

It has been observed that animals exposed to severe stress as for example stress of vaccination, or when high performance is demanded, suffer from fatigue, diarrhea, resistance to feed intake, anaemia etc. when they only are fed standard feed. In such cases it is obviously a need for additives or supplements to the feed. However, it is usually difficult to define what are the causes for the observed problems and thus which additive to use. There are known numerous additives and feed supplements, but none have proved to solve all the above problems. Some additives are primarily intended for increased growth of the animal while others claim to improve its health. Vitamin deficiencies might be part of the problem, but then one should understand why this occurs even when the feed is expected to contain sufficient amounts of vitamins.

A special problem has been observed on racing poultry, in particular pullets. Females over a year old are known as hens and younger females as pullets. A pullet becomes a hen when she begins to lay eggs at 16 to 20 weeks of age.

Pullet rearing is an extremely important stage in the bird's life as its bodyweight development and disease status will strongly influence the laying performance of the flock. A pullet is normally being administered around fifteen vaccines by various routes during the very first weeks of its life. Improving the immune response of birds throughout this period will enhance not only the level of antibodies carried by individual birds, helps to fight vaccinated infection and also other natural infections. This is potentially leading to better flock uniformity and a more immune competent flock at point of lay.

It is well known that vaccination causes decrease of the performance of chicken. In particular, vaccination causes reduction in feed intake and feed efficiency. Therefore vaccination has an enormous economic impact for the chicken producers.

It is therefore the object of the present invention to arrive at a new feed supplement that would improve health and performance in pullets, especially during stress conditions and when high performance where demanded.

It has now been found surprisingly that feed additive compositions as hereinafter defined improves
immune status,
bone and skeletal development,
flock uniformity,
growth and feed conversion of pullets.

The scope and special features of the invention are as defined by the attached claims.

The feed additive composition according to the present invention comprises canthaxanthin, at least one vitamin selected from the group consisting of vitamin C and vitamin E, a trace mineral, in particular selenium, and optionally a mixture of at least two compounds selected from the group consisting of thymol, eugenol, vanillin and y-terpinene.

The inventors have been able to demonstrate that a mixture of these active ingredients used in synergy and in combination exhibits, in totally unexpected manner, the effects sought by the present invention of improving digestibility, growth and bone development and of boosting the immune system of the pullets.

In a specific example it has been found that feed intake, weight gain and feed conversion ratio can be improved in poultry pullets by administering to the animals an effective amount of a feed supplement composition consisting of canthaxanthin, vitamin C and vitamin E, selenium and a mixture of essential oils comprising as main ingredients thymol and eugenol.

As used throughout the specification and claims, the following definitions apply.

The term feed conversion ratio is determined on the basis of a growth trial comprising a first treatment in which the composition according to the invention is added to the animal feed in a suitable concentration per kg feed, and a second treatment (control) with no addition of the composition to the animal feed.

As it is generally known, an improved FCR is lower than the control FCR. In particular embodiments, the FCR is improved (i.e. reduced) as compared to the control by at least 1.0% or 5%.

Canthaxanthin and the vitamins E and C are commercially available or can easily be prepared by a skilled person using processes and methods well-known in the prior art. For example vitamin E is available under the Trademark ROVIMIX® E50, vitamin C under the Trademark ROVIMIX® C and canthaxanthin under the Trademark CAROPHYLL®Red (all compounds are supplied by DSM Nutritional Products, Kaiseraugst, Switzerland).

Selenium may be obtained from any source, and a composition thereof may be prepared using convenient technology selenium (e.g. in inorganic form Na₂SeO₃ or as organic complex). Commercially available products are MICROGRAN®Se or SELSAF®2000.

The mixture of essential oils according to the invention are commercially available (for example under the Trademark CRINA®, supplied by DSM Nutritional Products, Kaiseraugst , Switzerland) or can easily be prepared by a skilled person using processes and methods well-known in the prior art. The mixtures of essential oils can be used in highly purified forms or in the form of natural available plant extracts or extract-mixtures.

The term “extract” as used herein includes compositions obtained by solvent extraction (which are also known as “extracted oils”), steam distillation (which are also known as “essential oils”) or other methods known to the skilled person. Suitable extraction solvents include alcohols such as ethanol.

By the expression “natural” is in this context understood a substance which consists of compounds occurring in nature and obtained from natural products or through synthesis. The natural substance may preferably contain at least two of the compounds as defined above as main ingredient and additionally other essential oil compounds as for example capsaicin, tannin or carvacrol.

In a first aspect, this invention relates to the use of canthaxanthin, at least one vitamin selected from the group consisting of vitamin C and vitamin E, a trace mineral, in particular selenium, and optionally a mixture of essential oils comprising at least two compounds selected from the group consisting of thymol, eugenol, vanillin and γ-terpinene for improving feed intake, weight gain, feed conversion ratio and/or bone development in pullets and for boosting the immune system of the animals.

This aspect encompasses also a method of feeding of an animal with a feed supplement composition comprising as main ingredients canthaxanthin, at least one vitamin selected from the group consisting of vitamin C and vitamin E, a trace mineral, in particular selenium, and optionally a mixture of essential oils comprising at least two compounds selected from the group consisting of thymol, eugenol, vanillin and γ-terpinene.

Canthaxanthin, the least one vitamin selected from the group consisting of vitamin C and vitamin E, the trace mineral selenium and the mixture of essential oils are suitably administered together with the feed. The term feed or feed composition means any compound, preparation, mixture, or composition suitable for, or intended for intake by an animal. The term feed as used herein comprises both solid and liquid feed as well as drinking fluids such as drinking water.

Particularly, the combination of ingredients according to the invention can be added as a formulated feed supplement composition directly to the regular animal feed or to a premix containing other minerals, vitamins, amino acids and trace elements which is added to regular animal feed and thorough mixing to achieve even distribution therein.

In a second aspect, a feed supplement composition is provided which comprises canthaxanthin, at least one vitamin selected from the group consisting of vitamin C and vitamin E, a trace mineral, in particular selenium, and optionally a mixture of essential oils comprising at least two compounds selected from the group consisting of thymol, eugenol, vanillin and y-terpinene.

A preferred feed supplement composition comprises canthaxanthin, vitamin C, vitamin E, the trace mineral selenium, and a mixture of essential oils with the main ingredients thymol and eugenol in amounts sufficient to reach the following concentrations in the final feed:
from about 2 to 100 ppm canthaxanthin
from about 20 to 200 ppm vitamin E
from about 20 to 200 ppm vitamin C
from about 0.01 to 0.5 ppm selenium
from about 20 to 250 ppm of an essential oil mixture.

According to the present invention it is further advantageous if the feed supplement composition according to the invention also contains one or more of the following ingredients: Vitamin A, Biotin, copper (e.g. as CuSO₄), zinc (e.g. as ZnSO₄), cobalt (e.g. as CoSO₄), iodine (e.g. as KI), manganese (e.g. as MnSO₄) and/or calcium (e.g. as CaSO₄).

A third aspect of the invention relates to a premix or regular animal feed which comprises a feed supplement composition according to the invention.

In the manufacture of poultry feed in accordance with the invention,
from about 2 to 100 ppm canthaxanthin, preferably 2 to 10 ppm
from about 20 to 200 ppm vitamin E, preferably 30 to 150 ppm
from about 20 to 200 ppm vitamin C, preferably 50 to 150 ppm
from about 0.01 to 0.5 ppm selenium, preferably 0.05 to 0.2 ppm
from about 20 to 250 ppm of an essential oil mixture, preferably 30 to 150 ppm are added to the regular poultry feed.

In a preferred embodiment of a poultry feeding concept, the active ingredients according to the invention are being used in the final regular poultry feed with the following amounts
2, 4 or 6 ppm canthaxanthin
35, 70 or 105 ppm vitamin E
50, 100 or 150 ppm vitamin C
0.05, 0.1 or 0.15 ppm selenium
34, 67 or 101 ppm of a mixture of essential oils.

Examples of particularly preferred mixtures of essential oils comprise
2% to 10% thymol, preferably 5%
10% to 20% eugenol, preferably 17%
0.5% to 5% y-terpinene, preferably 1%
5% to 10% vanillin, preferably 7%
55% to 82.5% fillers, carriers and emulsifying surfactants.

The mixture of essential oils includes optionally other chemical compounds, for example at least one additional compound selected from the following group (calculated in amounts as per kg of feed):
up to about 1 mg of propylidene, butylidene, phtalides, gingerol, lavender oil;
up to about 2 mg of deca-, undeca-, dodecalactones, ionones, irone, resorcinol, eucalyptol, menthol, peppermint oil, alpha-pinene;
up to about 3 mg of limonene, guajacol, anethol, linalool, methyl dihydrojasmonate;
up to about 4 mg of carvacrol, propionic, acetic or butyric acid, rosemary oil, clove oil, geraniol, terpineol, citronellol;
up to about 5 mg of amyl and/or benzyl salicylate, cinnamaldehyde, a plant polyphenol (tannin);
and up to about 5 mg of a powder of turmeric or of an extract of curcuma .

The emulsifying agent can be selected advantageously from those of a rather hydrophilic nature, for example among polyglycerol esters of fatty acids such as esterified ricinoleic acid or propylene glycol esters of fatty acids, saccharo-esters or saccharo-glycerides, polyethylene glycol, lecithins etc.

The incorporation of the feed supplement composition as exemplified herein above to poultry feeds is in practice carried out using a concentrate or a premix. A premix designates a preferably uniform mixture of one or more micro-ingredients with diluent and/or carrier. Premixes are used to facilitate uniform dispersion of micro-ingredients in a larger mix. A premix can be added to feed ingredients or to the drinking water as solids (for example as water soluble powder) or liquids.

Further, optional, feed-additive ingredients are aroma compounds; stabilisers; antimicrobial peptides; polyunsaturated fatty acids (PUFAs); reactive oxygen generating species; and/or at least one enzyme selected from amongst phytase (EC 3.1.3.8 or 3.1.3.26); xylanase (EC 3.2.1.8); galactanase (EC 3.2.1.89); alpha-galactosidase (EC 3.2.1.22); protease (EC 3.4., phospholipase A1 (EC 3.1.1.32); phospholipase A2 (EC 3.1.1.4); lysophospholipase (EC 3.1.1.5); phospholipase C (EC 3.1.4.3); phospholipase D (EC 3.1.4.4); amylase such as, for example, alpha-amylase (EC 3.2.1.1); and/or beta-glucanase (EC 3.2.1.4 or EC 3.2.1.6).

Examples of antimicrobial peptides (AMP's) are CAP18, Leucocin A, Protegrin-1, Thanatin, Defensin, Lactoferrin, Lactoferricin, and Ovispirin such as Novispirin (Robert Lehrer, 2000), Plectasins, and Statins.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Usually fat-and water-soluble vitamins, as well as trace minerals form part of a so-called premix intended for addition to the feed, whereas macro minerals are usually separately added to the feed.

A premix can contain, for example, per ton of poultry feed, 50 to 200 g of a propylene glycol solution of the mixture of the active compounds, 20 to 1000 g of an emulsifying agent, 50 to 900 g of cereals and by-products, 20 to 100 g of a proteinic support (milk powder, casein, etc.) and 50 to 300 g of a mineral component (expanded silica, feed quality lime, bi-calcium phosphate, etc.).

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

The invention is further explained in connection with the following example.

### EXAMPLE

1. Experimental Design

Global Design

The experiment concerns 4 groups of 260 pullets. Each group is divided into 10 repeats of 26 birds. Animals are placed in cages during rearing period.

Bodyweight measurements are planned at day-old and at 3, 8, 12 and 17 weeks of age. Feed intake is also calculated at the same ages. Thus, we can determine feed conversion ratio for each period.

Immune status is evaluated at 6, 12, 15 and 17 weeks of age.

Bone and skeletal development is estimated through tibia strength measurements coordinated at 3 different ages: 4, 8 and 17 weeks of age.

Experimental Groups

4 treatments are compared:
A batch: control with no supplement,
B batch: with feed supplement composition (FSC) in the feed (dosage of 2 kg/t),
C batch: with feed supplement composition (FSC) (dosage of 4 kg/t),
D batch: with feed supplement composition (FSC) (dosage of 6 kg/t).

| | B final | C final | D final |
|---|---|---|---|
| | dosage | dosage | dosage |
| | in feed | in feed | in feed |
| FSC comprises: | ppm | ppm | ppm |
| Vitamin E: ROVIMIX ® E50 | 35 | 70 | 105 |
| Vitamin C: ROVIMIX ® C | 50 | 100 | 150 |
| Canthaxanthin: CAROPHYLL ®Red | 2 | 4 | 6 |
| Selenium: SELSAF ®2000 | 0.05 | 0.1 | 0.15 |
| Essential oils: CRINA ®100.696 | 34 | 68 | 102 |

2. Management of the Birds During the Test

Vaccine Program

Vaccine program is attached to protocol (annex 1).

Feeding

Feeding program is divided into 3 periods:
From day-old to 3 weeks of age: Starter feed (PLD),
From 4 to 10 weeks of age: Grower feed (PLC),
From 11 to 17 weeks of age: Pullet feed (PLE).

Full description of different feeds used during rearing period is attached to protocol (annex 2).

Detail of feed composition is presented in table below:

| | Starter | Grower | Pullet |
|---|---|---|---|
| | 0-3 weeks | 4 to 10 weeks | 11 to 18 weeks |
| Kg feed/bird | 0.5 | 2.5 | 4 |

Composition %

| | | | |
|---|---|---|---|
| Corn | 41.6 | 35.8 | 35 |
| Wheat | 25 | 32.5 | 34 |
| wheat bran | 1.2 | 7.5 | 7.5 |
| Soya meal 48 | 27.5 | 20 | 19 |
| Carbonate | 0 | 0.65 | 1 |
| Bical Phosph | 1.65 | 1.3 | 1.25 |
| NaCl | 0.2 | 0.25 | 0.25 |
| sodium bicarbonate | 0.2 | 0.1 | 0.1 |
| methionine DL | 0.15 | 0.1 | 0.1 |
| Lysine Hcl 78 | 0.1 | 0 | |
| Choline premix 20% | 0.2 | 0.2 | 0.2 |
| Trace elements premix | 1 | 1 | 1 |
| Vitamin premix | 0.6 | 0.6 | 0.6 |
| FSC | 0-2-4-6 kg/t | 0-2-4-6 kg/t | 0-2-4-6 kg/t |

%

| | | | |
|---|---|---|---|
| protein | 18.5 | 16 | 16 |
| Fat | 3 | 2.5 | 2.5 |
| Fiber | 3.5 | 3.5 | 3.5 |
| methio | 0.45 | 0.37 | 0.36 |
| lysine | 1.04 | 0.79 | 0.77 |
| Ash | 6 | 6 | 6 |
| Na | 0.15 | 0.14 | 0.14 |
| Ca | 1 | 1.1 | 1.3 |
| Phosphorus | 0.6 | 0.6 | 0.6 |
| ME Kcal | 2845 | 2753 | 2749 |

mg/kg except vit A et D in International units

| | | | |
|---|---|---|---|
| Vitamin A | 7800 | 7800 | 7800 |
| Vitamine D3 | 3000 | 3000 | 3000 |
| Vitamin E | 15 | 15 | 15 |
| Vitamin B 1 | 1.26 | 1.26 | 1.26 |
| Vitamin B2 | 4.8 | 4.8 | 4.8 |
| Vitamin B6 | 2.4 | 2.4 | 2.4 |
| Vitamine B12 | 0.009 | 0.009 | 0.009 |
| Vitamin K3 | 2.4 | 2.4 | 2.4 |
| Niacin | 37.2 | 37.2 | 37.2 |
| Panthothenic acid | 8.7 | 8.7 | 8.7 |
| Folic acid | 0.72 | 0.72 | 0.72 |
| Biotin | 0.12 | 0.12 | 0.12 |
| Vitamin C | 12 | 12 | 12 |
| Choline | 400 | 400 | 400 |
| cobalt | 0.4 | 0.4 | 0.4 |
| Copper | 15 | 15 | 15 |
| Iodium | 1.5 | 1.5 | 1.5 |
| Iron | 65 | 65 | 65 |
| Manganese | 90 | 90 | 90 |
| Selenium | 0.2 | 0.2 | 0.2 |

The FSC is produced with wheat middlings as carrier.

3. Experimental Procedure and Traits Measured

Calendar of the Test

Data collection takes place between 1 and 123 day-old. The main events are listed below:

| Age (weeks) | Events |
|---|---|
| During all the | Mortality recording |
| rearing period | |
| Day 1 | Individual wingbanding |
| | Distribution of birds among repeats |
| | Individual bodyweight measurement |
| | Beginning of supplementation |
| Week 3 | Feed intake measurement |
| | Individual bodyweight measurement |
| Week 4 | Blood sampling (for oxidative test) |
| | Tibia sampling |
| Week 6 | Blood sampling (for ND IHA analyses) |
| Week 8 | Feed intake measurement |
| | Individual bodyweight measurement |
| | Tibia sampling |
| Week 9 | Blood sampling (for oxidative test) |
| Week 12 | Feed intake measurement |
| | Individual bodyweight measurement |
| | Blood sampling (for ND IHA) |
| Week 14 | Blood sampling (for oxidative test) |
| Week 15 | Blood sampling (for ND IHA analyses) |
| Week 16 | Blood sampling (for oxidative test) |
| Week 17 | Feed intake measurement |
| | Individual bodyweight measurement |
| | Blood sampling (for ND IHA analyses |
| | and IBD ELISA analyses) |
| | Tibia sampling |

Bone and Skeletal Development

Bone and skeletal development is characterized evaluating tibia strength.

Synergie 200 MTS compression machine allows to measure stiffness, maximum force and fracture force of the tibia. 20 left tibia per treatment are analysed at 3 different ages: 4, 8 and 17 weeks old.

4. Criteria to Estimate Effect of the Experimental Product
Bock/weight: Bodyweights (average and uniformity) are compared at 4 different ages: 3, 8, 12 and 17 weeks of age.
Feed consumption: Feed intakes are compared at 4 different ages: 3, 8, 12 and 17 weeks of age.
Feed Conversion Ratio is ratio between feed intake and bodyweight. Treatments can also be compared for this trait at 4 different ages: 3, 8, 12 and 17 weeks of age.
Immune status: Immune status is compared at 4 different ages: 6, 12, 15 and 17 weeks of age.
Bone and skeletal development: Tibia strengths (stiffness and fracture force) are compared at 3 different ages: 4, 8 and 17 weeks of age.

5. Statistical Analysis

Experimental Unit/Statistical Unit

Experimental unit is the repeat of 26 pullets.

Statistical unit is the bird or the cage.

Compatiblity of Initial Groups

Day-old chicks are randomly distributed among different repeats.

Furthermore, day-old bodyweight measurements inform us about comparability of initial groups for average and uniformity of bodyweights.

Statistical Tests

Alpha risk will be 5%.

SAS software will be used to run statistical tests.

Quantitative Traits Analysis

For quantitative traits as bodyweight, mathematical model is:
Xᵢⱼₖ=μ+αᵢ+
Eᵢⱼ
Xᵢⱼ=measured trait
μ=average
αᵢ=fixed effect of i^{th} treatment
Eᵢⱼ=residual

Constraints of above mathematical model are:
data from each population have to be normally distributed and to get same variance,
recordings are supposed to be independent.

Normality of the traits is tested with residuals of Kolmogorov-Smirnov test.

Evenness of variances is tested with Bartlett test.

Fixed effects are tested using variance analysis. Then, Fisher's LSD (Least Significant Difference test) is used to compare the treatment groups means.

Qualitative Traits Analysis

χ² test is used to compare proportions (mortality trait for example)

6. Results

Feeding animals with a feed supplement composition according to the invention shows significant effect on
average bodyweight: experimental treatments>control treatment,
feed conversion ratio: control treatments>experimental treatments,
tibia strength: experimental treatments>control treatment.

Increasing dosage does not improve the effects. Therefore the dosage B looks to be satisfactory in improving bodyweight, feed conversion and tibia strength.

Supplements have also a positive impact on immune status for Newcastle disease offering a better and more homogeneous protection for the birds of C and D treatments.

Mortality and Bodyweight Measurements

Bodyweight according to treatment at 1, 22, 56, 85 and 123 days of age

| Age (days) | | | Bodyweight (g) | | Mortality | | | |
|---|---|---|---|---|---|---|---|---|
| (weeks) | | Treatment | (CV %) | | rate (%) | Pullets # | Statistical analysis | |
| 1 | d | A | 34.1 ± 2.5 | (7.3) a | | 260 | Kolmogorov-Smirnov Test | NS¹ |
| | | B | 34.1 ± 2.9 | (8.4) a | | 260 | Bartlett Test | NS |
| | | C | 34.2 ± 2.6 | (7.6) a | | 260 | Variance analysis: | NS |
| | | D | 33.9 ± 2.5 | (7.3) a | | 260 | Treatment comparison | — |
| 22 | d | A | 167.6 ± 19.3 | (11.5) b | 0.8% | 258 | Kolmogorov-Smirnov Test | HS |
| (3 | wk) | B | 162.8 ± 21.6 | (13.3) a | 1.9% | 254 | Bartlett Test | NS |
| | | C | 167.8 ± 21.1 | (12.6) b | 0.8% | 258 | Variance analysis: | HS |
| | | D | 169.1 ± 21.4 | (12.6) b | 0.8% | 258 | Treatment comparison | B < A, C, D |
| 56 | d | A | 622.2 ± 67.4 | (10.8) a | 1.5% | 235² | Kolmogorov-Smirnov Test | HS |
| (8 | wk) | B | 634.6 ± 67.2 | (10.6) ab | 2.3% | 232 | Bartlett Test | NS |
| | | C | 640.7 ± 72.0 | (11.2) b | 1.2% | 237 | Variance analysis: | HS |
| | | D | 644.9 ± 66.9 | (10.4) b | 1.2% | 237 | Treatment comparison | A < C, D |
| 85 | d | A | 1056.6 ± 93.4 | (8.8) a | 1.5% | 216³ | Kolmogorov-Smirnov Test | NS |
| (12 | wk) | B | 1078.5 ± 94.1 | (8.7) b | 2.3% | 214 | Bartlett Test | NS |
| | | C | 1080.7 ± 94.7 | (8.8) b | 1.2% | 217 | Variance analysis: | HS |
| | | D | 1083.8 ± 86.7 | (8.0) b | 1.2% | 217 | Treatment comparison | A < B, C, D |
| 123 | d | A | 1479.5 ± 110.7 | (7.5) a | 1.5% | 216 | Kolmogorov-Smirnov Test | NS |
| (17 | wk) | B | 1524.8 ± 114.8 | (7.5) b | 2.3% | 214 | Bartlett Test | NS |
| | | C | 1520.2 ± 116.8 | (7.7) b | 1.2% | 217 | Variance analysis: | VHS |
| | | D | 1515.0 ± 107.6 | (7.1) b | 1.2% | 217 | Treatment comparison | A < D, C, B |

¹NS = Non significant, S = Significant difference (P < 0.05), HS = Highly Significant difference (P < 0.01), VHS = Very Highly Significant difference (P < 0.001)
²20 animals per treatment are killed at 27 d for taking tibia sample
³20 animals per treatment are killed at 56 d for taking tibia sample

Generally speaking, mortality rate is quite low for every treatment. It varies between 1.2% and 2.3% according to group. There are no statistical significant difference between treatments at 17 weeks-old (χ²: p=0.677).

At Day 1, average bodyweights of the different treatments are comparable (around 34 g). At day 22, B group shows lower bodyweight than the 3 other groups. Then, at the 3 other ages (56, 85 and 123 day-old), average bodyweights of the 3 experimental batches (B, C and D) are higher than the control group (A). Difference is around 40 g at 17 weeks-old ( [symbol] half a week of growth).

Flock uniformity is comparable for the 4 groups. Thus, Bartlett test that compares evenness of variances is not significant. At 123 day-old, bodyweight CV vary between 7.1 and 7.7% according to the group.

Daily Feed Consumption

Daily Feed Consumption accordina to treatment at 22. 56. 85 and 123 days of aae

| | | Feed Consumption | | | |
|---|---|---|---|---|---|
| Age (days) | Treatment | (g/bird/day) (CV %) | Cages # | Statistical analysis | |
| 22 d | A | 17.6 ± 1.0 (5.8) a | 10 | Kolmogorov-Smirnov Test | NS¹ |
| (3 wk) | B | 17.4 ± 0.8 (4.7) a | 10 | Bartlett Test | NS |
| | C | 17.2 ± 0.5 (3.0) a | 10 | Variance analysis: | NS |
| | D | 17.6 ± 1.1 (6.4) a | 10 | Treatment comparison | — |
| 56 d | A | 36.4 ± 0.6 (1.6) a | 10 | Kolmogorov-Smirnov Test | S |
| (8 wk) | B | 35.8 ± 1.0 (2.7) a | 10 | Bartlett Test | NS |
| | C | 36.2 ± 1.0 (2.7) a | 10 | Variance analysis: | NS |
| | D | 36.6 ± 1.1 (3.1) a | 10 | Treatment comparison | — |
| 85 d | A | 45.3 ± 0.5 (1.1) a | 10 | Kolmogorov-Smirnov Test | NS |
| (12 wk) | B | 46.0 ± 1.0 (2.1) a | 10 | Bartlett Test | NS |
| | C | 45.8 ± 0.9 (2.0) a | 10 | Variance analysis: | NS (p = 0.098) |
| | D | 46.3 ± 1.0 (2.1) a | 10 | Treatment comparison | — |
| 123 d | A | 53.8 ± 0.7 (1.3) a | 10 | Kolmogorov-Smirnov Test | NS |
| (17 wk) | B | 54.5 ± 1.0 (1.8) a | 10 | Bartlett Test | NS |
| | C | 53.8 ± 1.1 (2.0) a | 10 | Variance analysis: | NS |
| | D | 54.1 ± 1.0 (1.9) a | 10 | Treatment comparison | — |

¹NS = Non significant, S = Significant difference (P < 0.05), HS = Highly Significant difference (P < 0.01), VHS = Very Highly Significant difference (P < 0.001)

There is no statistical significant difference between the different groups for Daily Feed Consumption (DFC) trait. Average DFC is 54.0 g/bird/day at 123 day-old for the whole sample.

Feed Conversion Ratio

Feed Conversion Ratio according to treatment at 22. 56. 85 and 123 days of aae

| Age (days) | | Treatment | FCR (CV %) | Cages # | Statistical analysis | |
|---|---|---|---|---|---|---|
| 22 | d | A | 2.21 ± 0.14 (6.5) a | 10 | Kolmogorov-Smirnov Test | NS¹ |
| | | B | 2.25 ± 0.11 (4.8) a | 10 | Bartlett Test | NS |
| (3 | wk) | C | 2.15 ± 0.09 (4.1) a | 10 | Variance analysis: | NS |
| | | D | 2.19 ± 0.20 (9.0) a | 10 | Treatment Effect | — |
| 56 | d | A | 3.36 ± 0.15 (4.5) a | 10 | Kolmogorov-Smirnov Test | NS |
| (8 | wk) | B | 3.24 ± 0.09 (2.7) a | 10 | Bartlett Test | NS |
| | | C | 3.24 ± 0.12 (3.7) a | 10 | Variance analysis: | NS (p = 0.06) |
| | | D | 3.24 ± 0.10 (3.2) a | 10 | Treatment Effect | — |
| 85 | d | A | 3.74 ± 0.08 (2.2) a | 10 | Kolmogorov-Smirnov Test | NS (p = 0.06) |
| (12 | wk) | B | 3.72 ± 0.07 (1.8) a | 10 | Bartlett Test | NS |
| | | C | 3.69 ± 0.13 (3.4) a | 10 | Variance analysis: | NS |
| | | D | 3.72 ± 0.11 (3.0) a | 10 | Treatment Effect | — |
| 123 | d | A | 4.54 ± 0.06 (1.4) b | 10 | Kolmogorov-Smirnov Test | NS |
| (17 | wk) | B | 4.46 ± 0.08 (1.8) a | 10 | Bartlett Test | NS |
| | | C | 4.39 ± 0.08 (1.9) a | 10 | Variance analysis: | HS |
| | | D | 4.45 ± 0.10 (2.2) a | 10 | Treatment Effect | C, D, B < A |

¹NS = Non significant, S = Significant difference (P < 0.05), HS = Highly Significant difference (P < 0.01), VHS = Very Highly Significant difference (P < 0.001)

Bodyweights of dead birds (2 origins: mortality or tibia sampling at 27 or 56 days of age) are included to calculate Feed Conversion Ratio (FCR).

At 123 days-old, the 3 experimental groups (B, C and D) show better FCR than control group (A). Differences between treatments are not significant at younger ages (22, 56 and 85 days of age).

Immune Status

Newcastle Disease

Newcastle Disease antibodies titres according to treatment at 40. 81, 105 and 123 days of age

| Age (days) | | Log(titre) | | | | |
|---|---|---|---|---|---|---|
| (weeks) | Treatment | (CV %) | | Pullets # | Statistical analysis | |
| 40 d | A | 0.6 ± 0 | (0) | 20 | Kolmogorov-Smirnov Test | — |
| (6 wk) | B | 0.6 ± 0 | (0) | 20 | Bartlett Test | — |
| | C | 0.6 ± 0 | (0) | 20 | | |
| | D | 0.6 ± 0 | (0) | 20 | | |
| 81 d | A | 1.6 ± 0.4 | (25.0) | 19 | Kolmogorov-Smirnov Test | HS |
| (12 wk) | B | 1.6 ± 0.5 | (31.4) | 19 | Bartlett Test | NS |
| | C | 1.8 ± 0.6 | (34.8) | 20 | | |
| | D | 1.6 ± 0.5 | (31.4) | 20 | | |
| 105 d | A | 2.6 ± 0.7 | (28.2) | 19 | Kolmogorov-Smirnov Test | HS |
| (15 wk) | B | 2.7 ± 0.7 | (24.6) | 19 | Bartlett Test | NS |
| | C | 2.4 ± 0.6 | (26.6) | 20 | | |
| | D | 2.4 ± 0.9 | (36.0) | 20 | | |
| 123 d | A | 3.9 ± 0.4 | (11.1) | 18 | Kolmogorov-Smirnov Test | HS |
| (17 wk) | B | 3.8 ± 0.4 | (10.0) | 18 | Bartlett Test | NS (p = 0.098) |
| | C | 3.9 ± 0.2 | (6.0) | 20 | | |
| | D | 3.9 ± 0.3 | (8.5) | 20 | | |

¹NS = Non significant, S = Significant difference (P < 0.05), HS = Highly Significant difference (P < 0.01), VHS = Very Highly Significant difference (P < 0.001)

Newcastle Disease antihodies titres classes according to treatment at 123 days of age

| Age (days) | | Class | | | |
|---|---|---|---|---|---|
| (weeks) | Treatment | 0 | 1 | Pullets # | Chi2 |
| 123 d | A | 5 | 13 | 18 | NS¹ |
| (17 wk) | B | 5 | 13 | 18 | (p = 0.069) |
| | C | 0 | 20 | 20 | |
| | D | 3 | 17 | 20 | |

¹NS = Non significant, S = Significant difference (P < 0.05), HS = Highly Significant difference (P < 0.01), VHS = Very Highly Significant difference (P < 0.001)

At 40 days-old (before 1^{st} ND vaccination), no antibodies are detected.

Statistics are run on logarithm of titres. Normality of the trait is not accepted at any age then results of variance analysis are not presented.

Bartlett test is close to be significant at 123 day-old. Thus, titres of A and B treatments seems to show higher CV at this age.

To compare treatments at 123 day-old, we choose to create 2 titres categories:

Class 0: titre value<4096,

Class 1: titre value≧4096.

We use χ² test to compare proportions of birds in each class. Differences between groups are, here, close to be significant (p=0.069). With a larger sample size, we would probably get significant differences (p<0.05). Thus, A and B treatments would show a higher proportion of birds with lower antibodies titres values.

Infectious Bursal Disease

Infectious Bursal Disease antibodies titres accordina to treatment at 123 days of aae

| Age (days) | | | | | |
|---|---|---|---|---|---|
| (weeks) | Treatment | IBD Titre (CV %) | Pullets # | Statistical analysis | |
| 123 d | A | 4564 ± 616 (13.5) | 16 | Kolmogorov-Smirnov Test | NS (p > 0.15) |
| (17 wk) | B | 4686 ± 704 (15.0) | 18 | Bartlett Test | NS (p = 0.93) |
| | C | 4557 ± 609 (13.4) | 20 | Variance analysis: | NS (p = 0.90) |
| | D | 4540 ± 655 (14.4) | 20 | | |

¹NS = Non significant, S = Significant difference (P < 0.05), HS = Highly Significant difference (P < 0.01), VHS = Very Highly Significant difference (P < 0.001)

Immune status is not statistically different between treatments for IBD.

Bone and Skeletal Development

Fracture Force, Maximum Force, and Stiffness of tibia according to treatment at 27, 56 and 123 days of age

| Age (days) | | Fracture Force(N) | | Maximum Force (N) | | Stiffness (N/mm) | | |
|---|---|---|---|---|---|---|---|---|
| (weeks) | Treatment | (CV %) | | (CV %) | | (CV %) | | Pullets # |
| 27 d | A | 24.4 ± 13.4 | (55.0) | 50.7 ± 10.0 | (19.7) | 50.2 ± 10.1 | (20.1) | 20 |
| (4 wk) | B | 19.3 ± 6.9 | (35.9) | 49.4 ± 7.4 | (15.0) | 49.3 ± 9.1 | (18.4) | 20 |
| | C | 21.0 ± 14.1 | (67.3) | 47.4 ± 9.2 | (19.5) | 45.2 ± 10.4 | (23.0) | 20 |
| | D | 25.5 ± 15.0 | (58.9) | 52.3 ± 10.4 | (19.9) | 49.6 ± 12.4 | (25.1) | 20 |
| 56 d | A | 83.5 ± 44.0 | (52.7) | 132.5 ± 24.9 | (18.8) | 97.0 ± 21.0 | (21.7) | 19 |
| (8 wk) | B | 95.7 ± 49.2 | (51.4) | 145.6 ± 31.8 | (21.8) | 103.1 ± 18.5 | (18.0) | 20 |
| | C | 93.3 ± 44.8 | (48.0) | 141.4 ± 26.5 | (18.7) | 104.3 ± 20.2 | (19.4) | 20 |
| | D | 77.8 ± 44.9 | (57.7) | 134.1 ± 35.6 | (26.5) | 95.8 ± 25.6 | (26.7) | 20 |
| 123 d | A | 80.2 ± 41.3 | (51.4) | 173.8 ± 26.2 | (15.1) | 136.1 ± 17.1 | (12.5) | 20 |
| (17 wk) | B | 93.5 ± 47.2 | (50.5) | 191.4 ± 18.1 | (9.5) | 148.4 ± 17.6 | (11.9) | 20 |
| | C | 91.3 ± 42.8 | (46.8) | 180.2 ± 23.7 | (13.1) | 143.3 ± 17.0 | (11.9) | 20 |
| | D | 90.5 ± 58.7 | (64.9) | 188.7 ± 25.1 | (13.3) | 147.3 ± 18.2 | (12.4) | 20 |

¹NS = Non significant, S = Significant difference (P < 0.05), HS = Highly Significant difference (P < 0.01), VHS = Very Highly Significant difference (P < 0.001)

Statistical analysis for tibia strenath measurements at 27 days of aae

| | Fracture | Maximum | Stiffness |
|---|---|---|---|
| | Force(N) | Force (N) | (N/mm) |
| Kolmogorov-Smirnov | HS¹ | NS | NS |
| Test | | (p = 0.083) | (p = 0.095) |
| Bartlett Test | S | NS | NS |
| Variance analysis | NS | NS | NS |
| Treatment comparison | — | — | — |

¹NS = Non significant, S = Significant difference (P < 0.05), HS = Highly Significant difference (P < 0.01), VHS = Very Highly Significant difference (P < 0.001)

Statistical analysis for tibia strenath measurements at 56 days of aae

| | Fracture | Maximum | Stiffness |
|---|---|---|---|
| | Force(N) | Force (N) | (N/mm) |
| Kolmogorov-Smirnov | S¹ | NS | NS |
| Test | | | |
| Bartlett Test | NS | NS | NS |
| Variance analysis | NS | NS | NS |
| Treatment comparison | — | — | — |

¹NS = Non significant, S = Significant difference (P < 0.05), HS = Highly Significant difference (P < 0.01), VHS = Very Highly Significant difference (P < 0.001)

Statistical analysis for tibia strength measurements at 123 days of age

| | Fracture | Maximum | Stiffness |
|---|---|---|---|
| | Force(N) | Force (N) | (N/mm) |
| Kolmogorov-Smirnov | NS¹ | NS | NS |
| Test | | | |
| Bartlett Test | NS | NS | NS |
| Variance analysis | NS | NS | NS |
| | (p = 0.873) | (p = 0.080) | (p = 0.117) |
| Treatment comparison | — | — | — |

¹NS = Non significant, S = Significant difference (P < 0.05), HS = Highly Significant difference (P < 0.01), VHS = Very Highly Significant difference (P < 0.001)

Bodyweight and tibia weight of the pullets sacrificed for tibia strength evaluation at 27, 56 and 123 days of age

| Age (days) | | Bodyweight (g) | | Tibia weight (g) | | |
|---|---|---|---|---|---|---|
| (weeks) | Treatment | (CV %) | | (CV %) | | Pullets # |
| 27 d | A | 190 ± 21 | (10.9) | | | 20 |
| (4 wk) | B | 194 ± 21 | (10.6) | | | 20 |
| | C | 176 ± 17 | (9.6) | | | 20 |
| | D | 186 ± 32 | (17.0) | | | 20 |
| 56 d | A | 613 ± 74 | (12.1) | 5.2 ± 0.7 | (12.5) | 19 |
| (8 wk) | B | 623 ± 58 | (9.4) | 5.3 ± 0.6 | (12.0) | 20 |
| | C | 655 ± 60 | (9.2) | 5.3 ± 0.6 | (12.2) | 20 |
| | D | 620 ± 76 | (12.3) | 5.1 ± 0.8 | (15.2) | 20 |
| 123 d | A | 1476 ± 113 | (7.6) | 10.9 ± 0.9 | (7.8) | 20 |
| (17 wk) | B | 1558 ± 86 | (5.5) | 11.5 ± 0.7 | (6.4) | 20 |
| | C | 1490 ± 109 | (7.3) | 11.2 ± 0.7 | (6.0) | 20 |
| | D | 1517 ± 121 | (8.0) | 11.3 ± 0.5 | (4.6) | 20 |

¹NS = Non significant, S = Significant difference (P < 0.05), HS = Highly Significant difference (P < 0.01), VHS = Very Highly Significant difference (P < 0.001)

There is no significant difference between treatments for tibia fracture force at any age.

Tibia maximum force and stiffness differences are close to be significant (with, respectively, p=0.080 and p=0.117) at 123 days-old when they are not at younger ages (27 and 56 days-old). Thus, control group is characterized by weaker tibia compared to experimental batches. With a larger sample size, we would probably get significant differences (p<0.05). Higher values for the 3 experimental treatments are partially explained by higher bodyweights and tibia weights for these groups. Thus, introducing these traits in the mathematical model, we significantly affect p value:
Bodyweight as covariate in the model:
   Maximum force: p value for treatment effect=0.271,
   Stiffness: p value for treatment effect=0.368,
Tibia weight as covariate in the model:
   Maximum force: p value for treatment effect=0.265,
   Stiffness: p value for treatment effect=0.481.

Annex 1

| WEEK | DAY | VACCINES | DETAILS | Supplier | ADMINISTRATION | COMMENTS |
|---|---|---|---|---|---|---|
| 1 | 1 | MAREK (Rispens) | Rispens: This vaccine contains the | MERIAL (Sanofi | INJECTION | INACTIVATED |
| | | | Rispens CVI 988 strain of chicken herpes | group) | | VACCINE |
| | | | virus. This product is used as an aid in | | | |
| | | | the prevention of very virulent Marek's | | | |
| | | | disease in chickens. | | | |
| 1 | 1 | NOBILIS BI MA5 | A live vaccine for the immunization of | INTERVET | SPRAY | LIVE VACCINE |
| | | | chickens against Infectious Bronchitis | | Hatchery | |
| | | | serotype Massachusetts (strain Ma5) | | | |
| 1 | 1 | VAXXITEK HVT + IBD | This vaccine contains a live strain of | MERIAL | INJECTION | INACTIVATED |
| | | | serotype 3 vectored virus that has been | | | VACCINE |
| | | | shown to aid in the prevention of Bursal | | | |
| | | | disease and Marek's disease. This | | | |
| | | | vaccine is recommended for in ovo | | | |
| | | | vaccination of 18 to 19 day old | | | |
| | | | embryonated chicken eggs. | | | |
| 2 | | | | | | |
| 3 | 21 | POULVAC IB QX 2000 | Infectious bronchitis variant D388 | PFIZER | SPRAY | LIVE VACCINE |
| | | | (QX) | | | |
| 4 | | | | | | |
| 5 | 35 | NEMOVAC 1000 D | a freeze-dried lie vaccine against Swollen | MERIAL | SPRAY | LIVE VACCINE |
| | | | Head Syndrome prepared from a modified | | | |
| | | | live virus (PL21 strain) multiplied in VERO | | | |
| | | | cells. It is the first and only chicken- | | | |
| | | | derived live Avian Pneumovirus vaccine. | | | |
| | | | As an aid in the control of respiratory | | | |
| | | | disease in chickens associated with | | | |
| | | | Avian Pneumovirus | | | |
| 6 | 42 | BIORAL H 120 2000 D | Modified live vaccine against avian | MERIAL | SPRAY | LIVE VACCINE |
| | | | infectious bronchtis H 120 strain | | | |
| 6 | 43 | AVINEW 1000 DOSES | NewCastle Disease | MERIAL | SPRAY | LIVE VACCINE |
| 7 | | | | | | |
| 8 | 54 | POULVAC LARYNGO | LARYNGO TRACHEITIS INFECTIOUS | PFIZER | WATER | LIVE VACCINE |
| 9 | | | | | | |
| 10 | 70 | NEMOVAC 1000 D | SWOLLEN HEAD SYNDROME | MERIAL | SPRAY | LIVE VACCINE |
| 11 | 77 | NOBILIS BI 4 91 | live vaccine for the immunization of | INTERVET | SPRAY | LIVE VACCINE |
| | | | chickens against infectious bronchitis | | | |
| | | | serotype 4-91 and related serotypes. | | | |
| 12 | | | | | | |
| 13 | 91 | NOBILIS LA SOTA | Live vaccine for the immunization of | INTERVET | SPRAY | LIVE VACCINE |
| | | | chickens against Newcastle Disease | | | |
| 14 | 98 | POULVAC | Avian encephalomyelitis | PFIZER | WATER | LIVE VACCINE |
| | | ENCEPHALO | | | | |
| 15 | 106 | NOBILIS RT PONTE | EGG DROP SYNDROM, IB, RT, | INTERVET | INJECTION | INACTIVATED |
| | | | NEWCASTLE | | | VACCINE |

1. The use of canthaxanthin in combination with at least one vitamin selected from the group consisting of vitamin C and vitamin E, selenium and optionally a mixture of at least two compounds selected from the group consisting of thymol, eugenol, vanillin and γ-terpinene in poultry pullets feed for improving feed intake, weight gain, feed conversion ratio and/or bone development in pullets and/or for boosting the immune system of the animals.

2. The use according to claim 1 wherein canthaxanthin is combined with vitamin C, vitamin E, selenium and a mixture of essential oils comprising as main ingredients thymol and eugenol.

3. A feed supplement composition for pullets, comprising canthaxanthin, at least one vitamin selected from the group consisting of vitamin C and vitamin E, a trace mineral, in particular selenium, and optionally a mixture of essential oils comprising at least two compounds selected from the group consisting of thymol, eugenol, vanillin and γ-terpinene.

4. A feed supplement composition according to claim 3, comprising canthaxanthin, vitamin C, vitamin E, selenium and a mixture of essential oils comprising as main ingredients thymol and eugenol

5. A premix or regular animal feed which comprises a feed supplement composition according to claim 3.

6. An animal feed comprising
from about 2 to 100 ppm canthaxanthin, preferably 2 to 10 ppm
from about 20 to 200 ppm vitamin E, preferably 30 to 150 ppm
from about 20 to 200 ppm vitamin C, preferably 50 to 150 ppm
from about 0.01 to 0.5 ppm selenium, preferably 0.05 to 0.2 ppm
from about 20 to 250 ppm of a mixture of essential oils, preferably 30 to 150 ppm.

7. An animal feed according to claim 6 comprising
2, 4 or 6 ppm canthaxanthin
35, 70 or 105 ppm vitamin E
50, 100 or 150 ppm vitamin C
0.05, 0.1 or 0.15 ppm selenium
34, 67 or 101 ppm of a mixture of essential oils.

8. An animal feed according to claim 6 or 7 with a mixture of essential oils comprising
2% to 10% thymol, preferably 5%
10% to 20% eugenol, preferably 17%
0.5% to 5% y-terpinene, preferably 1%
5% to 10% vanillin, preferably 7%
55% to 82.5% fillers, carriers and emulsifying surfactants.

9. A method of feeding of an animal with a feed supplement composition comprising as main ingredients canthaxanthin, at least one vitamin selected from the group consisting of vitamin C and vitamin E, a trace mineral, in particular selenium, and optionally a mixture of essential oils comprising at least two compounds selected from the group consisting of thymol, eugenol, vanillin and γ-terpinene for improving feed intake, weight gain, feed conversion ratio and/or bone development in pullets and/or for boosting the immune system of the animals.
